(19) United States
(12) Patent Application Publication
(10) Pub. No.: US 2024/0122948 A1
(43) Pub. Date: Apr. 18, 2024

(54) COMPOSITIONS AND METHODS FOR PREVENTING AND PROTECTING AGAINST DAMAGE IN CELLS AND TISSUES

(71) Applicant: F3 PLATFORM BIOLOGICS, INC., Atlanta, GA (US)

(72) Inventors: David GRDINA, Naperville, IL (US); Jonathan PESKOFF, McLean, VA (US); Jonathan ROSEN, Dunwoody, GA (US)

(21) Appl. No.: 18/252,149

(22) PCT Filed: Nov. 22, 2021
(86) PCT No.: PCT/US2021/060291

§ 371 Date: May 8, 2023

Related U.S. Application Data
(60) Provisional application No. 63/118,333, filed on Nov. 25, 2020.
(60) Provisional application No. 63/118,341, filed on Nov. 25, 2020.
(60) Provisional application No. 63/118,344, filed on Nov. 25, 2020.
(60) Provisional application No. 63/118,347, filed on Nov. 25, 2020.
(60) Provisional application No. 63/118,352, filed on Nov. 25, 2020.
(60) Provisional application No. 63/118,356, filed on Nov. 25, 2020.
(60) Provisional application No. 63/118,362, filed on Nov. 25, 2020.
(60) Provisional application No. 63/118,369, filed on Nov. 25, 2020.
(60) Provisional application No. 63/118,373, filed on Nov. 25, 2020.
(60) Provisional application No. 63/118,378, filed on Nov. 25, 2020.
(60) Provisional application No. 63/118,382, filed on Nov. 25, 2020.
(60) Provisional application No. 63/118,388, filed on Nov. 25, 2020.
(60) Provisional application No. 63/118,391, filed on Nov. 25, 2020.
(60) Provisional application No. 63/118,396, filed on Nov. 25, 2020.
(60) Provisional application No. 63/118,400, filed on Nov. 25, 2020.
(60) Provisional application No. 63/118,378, filed on Nov. 25, 2020.

(51) Int. Cl. A61K 31/661; A61K 31/145

(52) U.S. Cl. CPC A61K 31/661; A61K 31/145

(57) ABSTRACT

Compositions for preventing and reversing ultraviolet (UV) radiation damage, preventing antimicrobial drug resistance, protecting a subject from acute radiation syndrome, reducing hair loss resulting from alopecia areata, reducing a probability of cervical cancer, reducing hair loss resulting from radiation therapy or chemotherapy, preventing complications arising from cosmic radiation exposure, preventing complications arising from a disease causing excess mucus formation, reducing a likelihood of infertility in cancer patients, preventing genomic instability from a diagnostic imaging or interventional radiology procedure, preventing radiation therapy-induced organ damage, preventing chemotherapy-associated peripheral neuropathy, protecting skin cells from radiation dermatitis, preventing radiation-induced fibrosis, and reducing ventilator-induced lung injury are provided. The composition includes a target compound. The target compound includes a phosphorothioate compound having one or more disulfide, mixed disulfide, or free thiol groups and a polyamine backbone with positively charged amine groups or an analog, derivative, prodrug, or salt thereof.

### CROSS-REFERENCE TO RELATED APPLICATIONS

The present application claims priority to and the benefit of the following United States Provisional Patent Applications: Serial Nos. 63/118,333; 63/118,341; 63/118,344; 63/118,347; 63/118,352; 63/118,356; 63/118,362; 63/118,369; 63/118,373; 63/118,378; 63/118,382; 63/118,388; 63/118,391; 63/118,396; and 63/118,400; all filed Nov. 25, 2020 and the contents of all of which as are hereby incorporated by reference herein in their entireties.

### FIELD

The presently-disclosed invention relates generally to compositions and methods for preventing and protecting against cellular and tissue damage, and more particularly to compositions and methods using certain phosphorothioate compounds to prevent and protect against various forms of genomic, cellular, and tissue damage.

### BACKGROUND

The human body has approximately 37 trillion cells that are constantly dividing, and even with a very low error rate, each cell could accumulate somewhere in the order of ten mutations each time it divides. As a result, trillions of mutations may occur every day. Other organisms, including microbes, experience similar mutations, although on a smaller scale. These mutations, as well as genomic instability, increase dramatically as a result of environmental factors, including exposure to ionizing and non-ionizing radiation (e.g., via UV light, medical procedures, etc.), viral illness, and the like. Most mutations are harmless or have no real effects, but occasionally mutations may have noticeable negative effects (e.g., somatic and germ line mutations and cancer).

Accordingly, there exists a need for both preventative and protective treatments to reduce or even inhibit genomic instability, mutations, and their resulting negative effects.

### BRIEF SUMMARY

One or more embodiments of the invention may address one or more of the aforementioned problems. In one aspect, a composition for preventing and reversing ultraviolet (UV) radiation damage is provided. In accordance with certain embodiments, the composition may include a target compound and a sunscreen. In some embodiments, the target compound may comprise a phosphorothioate compound having one or more disulfide, mixed disulfide, or free thiol groups and a polyamine backbone with positively charged amine groups or an analog, derivative, prodrug, or salt thereof.

In another aspect, a method of preventing and reversing UV radiation damage is provided. In accordance with certain embodiments, the method may include applying a composition to skin. In some embodiments, the composition may comprise a target compound and a sunscreen. In further embodiments, the target compound may comprise a phosphorothioate compound having one or more disulfide, mixed disulfide, or free thiol groups and a polyamine backbone with positively charged amine groups or an analog, derivative, prodrug, or salt thereof.

### DETAILED DESCRIPTION

The invention now will be described more fully hereinafter with reference to the accompanying drawings, in which some, but not all embodiments of the inventions are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. As used in the specification, and in the appended claims, the singular forms “a”, “an”, “the”, include plural referents unless the context clearly dictates otherwise.

### I. Definitions

As used herein, the term “composition” may comprise any composition containing one or more target compounds, including analogs, derivatives, prodrugs, or salts thereof. The target compounds may comprise target phosphorothioate compounds, target thiol-containing compounds, and/or redox-cycling nitroxide compounds. For example, the target phosphorothioate compounds may include those having a polyamine backbone with positively charged amine groups and/or disulfide, mixed disulfide, or free thiol groups. In some embodiments, for instance, the target phosphorothioate compounds include both a polyamine backbone having positively charged amine groups and one or more disulfide, mixed disulfide, or free thiol groups. Examples of target phosphorothioate compounds include, but are not limited to, S-2-(3-aminopropylamino)ethyl phosphorothioic acid (amifostine, WR-2721), 2-[(aminopropyl)amino]ethanethiol (amifostine free thiol form, WR-1065), [2-[(aminopropyl)amino]ethanethiol]N,N,′-dithiodi-2,1-(ethanediyl)bis-1,3-propanediamine (amifostine di sulfide form, WR-33278), S-[2-(3-methylaminopropyl)aminoethyl]phosphorothioate acid (phosphonol, WR-3689), 2-[3-(methylamino) propylamino] ethanethiol (WR-255591), S-3-(3-methylaminopropylamino) propyl phosphorothioic acid (WR-151327), and 3-(3-methylaminopropylamino) propanethiol dihydrochloride (WR-151326). The target thiol-containing compounds, for example, may include, but are not limited to, one or more of cysteine, cystamine, and cysteamine. The target redox-cycling nitroxide compounds, for instance, may include, but are not limited to, 4-hydroxy-2,2,6,6-tetramethylpiperidin-1-oxyl (i.e. 4-Hydroxy-TEMPO, or TEMPOL).

The term “salts”, as used herein, may refer to any acid or base salt, pharmaceutically acceptable solvates, or any complex of the compound that, when administered to a recipient, is capable of providing (directly or indirectly) a compound as described herein. It should be appreciated, however, that salts that are not pharmaceutically acceptable also lie within the scope of the invention. The preparation of salts can be carried out using known methods. For example, pharmaceutically acceptable salts of compounds contemplated herein as being useful may be synthesized by conventional chemical methods using a parent compound containing a base or an acid functionality. Generally, such salts may be prepared, for example, by making free acid or base forms of the compound and reacting with a stoichiometric quantity of the appropriate base or acid in water or in an organic solvent, or in a mixture of the two. Generally, non-aqueous media such as one or more of solvents such as ether, ethyl acetate, ethanol, isopropanol, or acetonitrile may be utilized. Examples of acid addition salts include one or more of mineral acid addition salts such as hydrochloride, hydrobromide, hydroiodide, sulphate, phosphate, and organic acid addition salts such as one or more of acetate, maleate, fumarate, citrate, oxalate, succinate, tartrate, malate, mandelate, methanesulphonate, and p-toluene-sulphonate. Examples of base addition salts include one or more of inorganic salts such as sodium, potassium, calcium, ammonium, magnesium, and lithium salts, and organic base salts such as one or more of ethylenediamine, ethanolamine, N,N-dialkyl-ethanolamine, triethanolamine, and basic amino acid salts.

The term “prevention” or any variation thereof, as used herein, may refer to inhibiting or reducing the probability of development of stochastic negative effects on the body, i.e. those negative effects having no threshold and where increasing doses or exposures do not result in greater severity of negative effects but rather result in a higher probability of a negative effect occurring.

The term “protection” or any variation thereof, as used herein, may refer to inhibiting the development of deterministic negative effects on the body, i.e. those negative effects whose severity increases as a function of increased exposure.

The terms “inhibit”, “reduce”, “decrease”, “diminish”, “lower”, “minimize”, or any variation of these terms, as used herein, may refer to any measurable decrease or complete inhibition to achieve a desired result. By way of example only, these terms may refer to at least a 10% change in a target (e.g., damage), with greater percentage changes being preferred. For instance, the change may also be greater than 25%, 35%, 45%, 55%, 65%, 75%, 85%, 95%, 96%, 97%, 98%, 99%, or other increments greater than 10%.

For ease of reference, the present invention will be described in terms of administration to human subjects. It will be understood, however, that such descriptions are not limited to administration to humans but will also include administration to other animal unless explicitly stated otherwise.

### II. Amifostine and Amifostine Analog Compositions

Certain embodiments according to the invention provide compositions and methods for preventing and/or protecting against damage comprising amifostine and/or one or more amifostine analogs.

A. Active Ingredients

According to certain embodiments, for instance, the active ingredient may comprise one or more phosphorothioates. In some embodiments, for example, the active ingredient may comprise one or more phosphorothioates having a polyamine backbone with positively charged amine groups and/or disulfide, mixed disulfide, or free thiol groups. In further embodiments, for instance, the active ingredient may comprise one or more phosphorothioates with both a polyamine backbone having positively charged amine groups and one or more disulfide, mixed disulfide, or free thiol groups.

In this way, and without being bound by theory, the positively charged amine groups of the polyamine backbone are attracted to DNA and brings thiol groups close to the DNA. The thiol groups scavenge free radicals and cause the free radicals to migrate away from the DNA backbone. As such, the free thiol forms of the target phosphorothioate compounds are likely the most active forms against free radical generation. Because free radicals are largely to blame for ionizing radiation-induced cell damage and/or death, the thiol groups in the target phosphorothioate compounds are invaluable in cytoprotection. Indeed, it is theorized that the thiol groups in particular reduce damage to DNA so that the DNA repair system is not overloaded and can efficiently repair DNA damage so a cell can survive.

Moreover, in addition to bringing the thiol groups closer to the DNA, the polyamine backbone may also enhance the fidelity of DNA repair, thereby preventing genomic instability. Specifically, without being bound by theory, the polyamine backbone may enhance the repair-signaling process by stabilizing the DNA strand and allowing proteins that identify damage, migrate to and collect damage, and/or the like to efficiently repair the DNA strand.

In some embodiments, for example, the active ingredient may comprise amifostine and/or its analogs, namely one or more of S-2-(3-aminopropylamino)ethyl phosphorothioic acid (amifostine, WR-2721), 2-[(aminopropyl)amino]ethanethiol (amifostine free thiol form, WR-1065), [2-[(aminopropyl) amino] ethanethiol] N,N,′-dithiodi-2,1-(ethanediyl)bis-1,3-propanediamine (amifostine disulfide form, WR-33278), S-[2-(3-methylaminopropyl)aminoethyl]phosphorothioate acid (phosphonol, WR-3689), 2-[3-(methylamino) propylamino] ethanethiol (WR-255591), S-3-(3-methylaminopropylamino) propyl phosphorothioic acid (WR-151327), and 3-(3-methylaminopropylamino) propanethiol dihydrochloride (WR-151326).

In certain embodiments, for instance, the active ingredient may comprise one or more thiol-containing compounds. By way of example only, such thiol-containing compounds may include, but are not limited to, one or more of cysteine, cystamine, and cysteamine. Without being limited by theory, the target thiol-containing compounds may provide cytoprotection via free radical scavenging by the thiol groups, DNA structural stabilization through polyamine-like electrostatic binding, and transient increased cell cycle progression times to allow for more time for repair prior to cell division through the modification and/or inhibition of proliferating associated proteins and enzymes such as topoisomerases.

In some embodiments, for example, the active ingredient may comprise one or more redox-cycling nitroxide compounds. By way of example only, such redox-cycling nitroxide compounds may include, but are not limited to, 4-hydroxy-2,2,6,6-tetramethylpiperidin-1-oxyl (i.e. 4-Hydroxy-TEMPO, or TEMPOL).

As previously described herein, according to certain embodiments, the composition may comprise a salt of one or more of the target phosphorothioate or thiol-containing compounds. Pharmaceutically acceptable salts, e.g., non-toxic, inorganic and organic acid addition salts, are known in the art. Exemplary salts include, but are not limited to, 2-hydroxyethanesulfonate, 2-naphthalenesulfonate, 3-hydroxy-2-naphthoate, 3-phenylpropionate, acetate, adipate, alginate, amsonate, aspartate, benzenesulfonate, benzoate, besylate, bicarbonate, bisulfate, bitartrate, borate, butyrate, calcium edetate, camphorate, camphorsulfonate, camsylate, carbonate, citrate, clavulariate, cyclopentanepropionate, digluconate, dodecylsulfate, edetate, edisylate, estolate, esylate, ethanesulfonate, finnarate, gluceptate, glucoheptanoate, gluconate, glutamate, glycerophosphate, glycollylarsanilate, hemi sulfate, heptanoate, hexafluorophosphate, hexanoate, hexylresorcinate, hydrabamine, hydrobromide, hydrochloride, hydroiodide, hydroxynaphthoate, iodide, isothionate, lactate, lactobionate, laurate, lauryl sulphonate, malate, maleate, mandelate, mesylate, methanesulfonate, methylbromide, methylnitrate, methylsulfate, mucate, naphthylate, napsylate, nicotinate, nitrate, N-methylglucamine ammonium salt, oleate, oxalate, palmitate, pamoate, pantothenate, pectinate, persulfate, phosphate, phosphateldiphosphate, picrate, pivalate, polygalacturonate, propionate, p-toluenesulfonate, saccharate, salicylate, stearate, subacetate, succinate, sulfate, sulfosaliculate, suramate, tannate, tartrate, teoclate, thiocyanate, tosylate, triethiodide, undecanoate, and valerate salts, and the like.

B. Dosage Forms

The composition may comprise a number of dosage forms. According to certain embodiments, for example, the composition may comprise a topical preparation. In such embodiments, for instance, topical preparations may include creams, ointments, lotions, gels, patches, tapes, sponges, vapors, powders, solutions, emulsions, liposome suspensions, foams, pastes, solids, tinctures, suppositories, and/or the like as understood by one of ordinary skill in the art. Topical preparations may be configured for cutaneous administration, transdermal administration, rectal administration, vaginal administration, ocular administration, otic administration, and/or the like as understood by one of ordinary skill in the art.

In other embodiments, for example, the composition may comprise an oral preparation. For purposes of this disclosure, oral preparations may also include preparations configured for sublingual or buccal administration. In such embodiments, for instance, the oral preparation may be solid or liquid. For example, in some embodiments, solid oral preparations may include tablets, including solid tablets, chewable tablets, quick dissolve tablets, film-coated tablets, enteric-coated tablets, effervescent tablets, multi-layer tablets, and bi-layer tablets; lozenges, including chewable lozenges; capsules, including hard capsules and soft capsules; caplets; powders, including reconstitutable powders, granules, dispersible granules, particles, and microparticles; cachets; beads and/or the like as understood by one of ordinary skill in the art. In other embodiments, for instance, liquid oral preparations may include drops, syrups, elixirs, spirits, tinctures, suspensions, solutions, and/or the like as understood by one of ordinary skill in the art. According to certain embodiments, for example, the oral preparation may be configured for immediate action/release. In other embodiments, however, the oral preparation may be configured for prolonged action/sustained release.

In further embodiments, for instance, the composition may comprise a subcutaneous preparation. In certain embodiments, for example, the composition may comprise an intramuscular preparation. In some embodiments, for instance, the composition may comprise an intravenous (IV) preparation. In other embodiments, for example, the composition may comprise an intradermal preparation. In further embodiments, for instance, the composition may comprise an intrathecal preparation. In such embodiments, for example, any of the subcutaneous preparations, intramuscular preparations, IV preparations, intradermal preparations, and intrathecal preparations may comprise a solution or a colloid.

In additional embodiments, for instance, the composition may comprise an inhalable preparation. In such embodiments, for example, the composition may comprise an aerosol, dry powder, or atomized solution configured for use in an inhaler (e.g., a meter-dosed inhaler (MDI), a dry powder inhaler (DPI), or a soft mist inhaler (SMI)), nebulizer, or similar device as understood by one of ordinary skill in the art. In certain embodiments in which the composition is an aerosol, the composition may be used in conjunction with any standard propellant (e.g., hydrofluoroalkane (HFA)), as understood by one of ordinary skill in the art. Alternatively, the composition may comprise an atomized solution configured for intranasal preparation. For example, in such embodiments, the atomized particles may be larger than those use in inhalable preparations configured for use in an inhaler or nebulizer.

C. Dosage

In accordance with certain embodiments, the dosage of the composition administered to a subject may depend on the ultimate target of the composition. For example, in some embodiments, the goal of administering the composition may be to prevent genomic instability from occurring over a period of time (e.g., from UV radiation, antimicrobial drug use, viral exposure, etc.). In other embodiments, for instance, the goal of administering the composition may be for cytoprotection, but the composition may need to be administered multiple times (e.g., 20-30) over a relatively short period. In such embodiments, for instance, a relatively low dose (e.g., from about 200 mg/m² to about 350 mg/m² per dose). may be used to either prevent genomic instability or to protect cells from cell death and/or damage while also avoiding cumulative toxicity and minimizing the risks of simultaneously protecting tumor metastases. In some embodiments in which the goal of administering the composition may be to prevent genomic instability, a dose from about 10 mg/m² to about 200 mg/m² may be used. In further embodiments, for instance, a dose from about 75 mg/m² to about 150 mg/m² may be used. In this regard, for example, the dose used may be from at least about any of the following: 10, 25, 50, 75, 100, 125, 150, 175, 180, 185, 190, 195, 196, 197, 198, and 199 mg/m² and/or at most about 200, 175, 150, 125, 100, 75, 50, 45, 40, 35, 30, 25, 20, 15, 14, 13, 12, and 11 mg/m² (e.g., about 100-150 mg/m², about 50-199 mg/m², etc.).

In other embodiments, however, the goal of administering the composition may be to protect cells from cell death (e.g., via high radiation exposure). In such embodiments, for example, the highest possible dose that can be tolerated may be administered. For instance, in such embodiments, a dose from about 200 mg/m² to about 2200 mg/m² may be used. In further embodiments, for example, a dose from about 400 mg/m² to about 1200 mg/m² may be used. In this regard, for instance, the dose used may be from at least about any of the following: 200, 300, 400, 500, 600, 700, 800, 900, 1000, 1100, 1200, 1300, 1400, 1500, 1600, 1700, 1800, 1900, 2000, 2100, 2150, 2160, 2170, 2180, 2190, 2195, 2196, 2197, 2198, and 2199 mg/m² and/or at most about 2200, 2100, 2000, 1900, 1800, 1700, 1600, 1500, 1400, 1300, 1200, 1100, 1000, 900, 800, 700, 600, 500, 400, 300, 250, 240, 230, 220, 210, 205, 204, 203, 202, and 201 mg/m² (e.g., about 700-1000 mg/m², about 200-900 mg/m², etc.). For example, lower doses within these ranges (e.g., 200-600 mg/m²) may be tolerated in multiple doses without incurring significant cumulative toxicity. Higher doses, however, may only be appropriate for single dose use. For instance, the highest dose typically used in an adult human may be about 740 mg/m². The maximum tolerated dose in an adult human may be about 1150 mg/m², and the maximum tolerated human pediatric dose may be about 2200 mg/m². Without being bound by theory, it is believed that changes in metabolism that occur during puberty may decrease the maximum tolerated dose from childhood to adulthood.

Moreover, in certain embodiments, the dosage of the composition administered to a subject may depend on the dosage form of the composition. For example, in some embodiments, the composition may be prepared for systemic administration (e.g., injection, oral preparation, systemic topical preparation, systemic inhalable preparation). In such embodiments, for instance, a relatively low dose may be used (e.g., about 25 mg/m² to about 200 mg/m²) for at least the reasons discussed previously herein.

In other embodiments, however, the composition may be prepared for local topical administration. In such embodiments, the dosage will depend upon the target or goal in administering the composition, as discussed above. For example, if the goal is to prevent genomic instability over a period of time or provide cytoprotection in a dose that can be given repeatedly, a relatively low dose may be used (e.g., about 10-200 mg/m² for genomic instability mutagenesis and cancer prevention and about 200-350 mg/m² per dose in a multiple dosing protocol for cytoprotection). However, if the goal is to protect cells from cell death, the highest possible dose that can be tolerated may be administered (e.g., 200-2200 mg/m²).

In accordance with certain embodiments, the timing of the administration of the dosage may depend on the goal of using the composition. In some embodiments in which the goal is to prevent genomic instability, the composition may be administered up to about 24 hours before exposure (e.g., to radiation) and up to about 24 hours after exposure. In further embodiments in which the goal is to prevent genomic instability, the composition may be administered up to about 12 hours before exposure and up to about 12 hours after exposure. In certain embodiments in which the goal is to prevent genomic instability, the composition may be administered up to about 3 hours before exposure and up to about 10 hours after exposure. In further embodiments in which the goal is to prevent genomic instability, the composition may be administered up to about 1 hour before exposure and up to 6 hours after exposure. In even further embodiments in which the goal is to prevent genomic instability, the composition may be administered up to about 30 minutes before exposure and up to 3 hours after exposure. In other embodiments in which the goal is cytoprotection, the composition must be administered before, and be present during, exposure. In this way, in certain embodiments, the composition may be administered about 15-30 minutes prior to exposure. Under conditions of the phosphorothioate and/or thiol induced delayed cytoprotective effect whereby the endogenous antioxidant enzyme manganese superoxide dismutase (MnSOD) is induced, the composition may be administered from about 6 to about 36 hours before radiation exposure to allow for maximum induction and presence of elevated MnSOD intracellular levels.

D. Additives

The composition may include one or more additives dependent upon the dosage form and intended treatment target of the composition.

1. Drug Additives

According to certain embodiments, for instance, the composition may include one or more additional drugs. In some embodiments, for example, the composition may include a beta-glucan. Without being limited by theory, beta-glucan has radioprotective properties and, when administered in conjunction with the target compound, may reduce the dosage of the target compound needed for cytoprotection or to prevent genomic instability. In other embodiments, for instance, the composition may include one or more of captopril, misoprostol, metformin, prostaglandins, and/or the like. In further embodiments, for example, the composition may include one or more antimicrobials, including one or more antibiotics, antivirals, antifungals, and/or antiparasitics.

For example, in some embodiments the composition may include one or more antibiotics. Such antibiotics may include, but are not limited to, vancomycin, teicoplanin, linezolid, daptomycin, sulfamethoxazole, doxycycline, ceftobiprole, ceftaroline fosamil, clindamycin, dalbavancin, fusidic acid, mupirocin, omadacycline, oritavancin, tedizolid, telavancin, tigecycline, amikacin, gentamicin, kanamycin, neomycin, netilimicin, tobramycin, paromomycin, streptomycin, spectinomycin, geldanamycin, herbimycin, rifaximin, loracarbef, ertapenem, doripenem, imipenem (with or without cilastatin), meropenem, ceftazidime, cefepime, ceftolozane (with or without tazobactam), fluoroquinolones, piperacillin (with or without tazobactam), ticarcillin (with or without clavulanic acid), streptogramins, cefadroxil, cefazolin, cephradine, cephapirin, cephalothin, cefalexin, cefaclor, cefoxitin, cefotetan, cefamandole, cefmetazole, cefonicid, cefprozil, cefuroxime, cefixime, cefdinir, cefditoren, cefoperazone, cefotaxime, cefpodoxime, ceftibuten, ceftizoxime, moxalactam, ceftriaxone, lincomycin, azithromycin, clarithromycin, erythromycin, roxithromycin, telithromycin, spiramycin, fidaxomicin, aztreonam, furazolidone, nitrofurantoin, posizolid, radezolid, amoxicillin (with or without clavulanate), ampicillin (with or without sulbactam), azlocillin, dicloxacillin, flucloxacillin, mezlocillin, methicillin, nafcillin, oxacillin, penicillin G, penicillin V, temocillin, ticarcillin (with or without clavulanate), bacitracin, colistin, polymyxin B, ciprofloxacin, enoxacin, gatifloxacin, gemifloxacin, levofloxacin, lomefloxacin, moxifloxacin, nadifloxacin, nalidixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethizole, sulfanilamide, sulfasalazine, sulfisoxazole, sulfonamidochrysoidine, demeclocycline, metacycline, minocycline, oxytetracycline, tetracycline, clofazimine, dapsone, capreomycin, cycloserine, ethambutol, ethionamide, isoniazid, pyrazinamide, rifampicin (also known as rifampin), rifabutin, rifapentine, arsphenamine, chloramphenicol, fosfomycin, metronidazole, platensimycin, quinupristin, dalfopristin, thiamphenicol, tinidazole, trimethoprim, teixobactin, malacidins, halicin, and other antiobiotics known in the art.

In further embodiments, for instance, the composition may include one or more antivirals. Such antivirals may include, but are not limited to, abacavir, acyclovir, adefovir, amantadine, ampligen, amprenavir, umifenovir, atazanavir, atripla, baloxavir marboxil, biktarvy, boceprevir, bulevirtide, cidofovir, cobicistat, combivir, daclatasvir, darunavir, delavirdine, descovy, didanosine, docosanol, dolutegravir, doravirine, edoxudine, efavirenz, elvitegravir, emtricitabine, enfuvirtide, entecavir, etravirine, famciclovir, fomivirsen, fosamprenavir, foscarnet, ganciclovir, ibacitabine, ibalizumab, idoxuridine, imiquimod, imunovir, indinavir, lamivudine, letermovir, lopinavir, loviride, maraviroc, methisazone, moroxydine, nelfinavir, nevirapine, nexavir, nitazoxanide, norvir, oseltamivir, penciclovir, peramivir, penciclovir, pleconaril, podophyllotoxin, raltegravir, remdesivir, ribavirin, rilpivirine, rimantadine, ritonavir, saquinavir, simeprevir, sofosbuvir, stavudine, taribavirin, telaprevir, telbivudine, tenofovir alafenamide, tenofovir disoproxil, tenofovir, tipranavir, trifluridine, trizivir, tromantadine, Truvada, umifenovir, valaciclovir, valganciclovir, vicriviroc, vidarabine, zalcitabine, zanamivir, zidovudine, and other antivirals known in the art.

In some embodiments, for example, the composition may include one or more antifungals (antimycotics). Such antifungals may include, but are not limited to, amphotericin B, candicidin, filipin, hamycin, natamycin, nystatin, rimocidin, bifonazole, butoconazole, clotrimazole, econazole, fenticonazole, isoconazole, ketoconazole, luliconazole, miconazole, omoconazole, oxiconazole, sertaconazole, sulconazole, tioconazole, albaconazole, efinaconazole, epoxiconazole, fluconazole, isavuconazole, itraconazole, posaconazole, propiconazole, ravuconazole, terconazole, voriconazole, abafungin, amorolfine, butenafine, naftifine, terbinafine, anidulafungin, caspofungin, micafungin, aurones, benzoic acid, ciclopirox, flucytosine, 5-fluorocytosine, griseofulvin, haloprogin, tolnaftate, undecylenic acid, triacetin, crystal violet, carbol fuchsin, orotomide, miltefosine, potassium iodide, nikkomycin, coal tar, copper(II) sulfate, selenium disulfide, sodium thiosulfate, piroctone olamine, iodoquinol, clioquinol, acrisorcin, zinc pyrithione, sulfur, and other antifungals known in the art.

In further embodiments, for instance, the composition may include one or more antiparasitics. Such antiparasitics may include, but are not limited to, nitazoxanide, melarsoprol, eflornithine, metronidazole, tinidazole, miltefosine, mebendazole, pyrantel pamoate, thiabendazole, diethylcarbamazine, ivermectin, niclosamide, praziquantel, albendazole, rifampin, amphotericin B, fumagillin, bephenium, piperazine, pyrvinium, flubendazole, and other antiparasitics known in the art.

In further embodiments, for instance, the composition may include one or more steroids. Such steroids may include clobetasol-17 propionate, betamethasone dipropionate, halobetasol propionate, diflorasone diacetate, fluocinonide, halcinonide, amcinonide, desoximetasone, triamcinolone acetonide, mometasone furoate, fluticasone propionate, halometasone, fluocinolone acetonide, hydrocortisone 17-valerate, hydrocortisone 17-butyrate, flurandrenolide, desonide, alclometasone dipropionate, hydrocortisone, betamethasone valerate, diflucortolone valerate, methylprednisolone aceponate, clobetasone 17-butyrate, hydrocortisone acetate, cortisone acetate, tixocortol pivalate, prednisolone, methylprednisolone, prednisone, triamcinolone alcohol, budesonide, betamethasone, betamethasone sodium phosphate, dexamethasone, dexamethasone sodium phosphate, fluocortolone, prednicarbate, fluocortolone caproate, fluocortolone pivalate, fluprednidene acetate, and other steroids known in the art.

2. Other Additives

According to certain embodiments, for example, the composition may include a wide range of materials including, but not limited to, diluents and bulking substances, binders and adhesives, lubricants, glidants, plasticizers, disintegrants, carrier solvent, buffers, colorants, flavorings, sweeteners, preservatives and stabilizers, and other pharmaceutical additives known in the art.

Diluents increase the bulk of a dosage form and may make the dosage form easier to handle. Exemplary diluents include, but are not limited to, lactose, dextrose, saccharose, cellulose, starch, and calcium phosphate for solid dosage forms, e.g., tablets and capsules; olive oil and ethyl oleate for soft capsules; water and vegetable oil for liquid dosage forms, e.g., suspensions and emulsions. Additional suitable diluents include, but are not limited to, sucrose, dextrates, dextrin, maltodextrin, microcrystalline cellulose (e.g., Avicel®), microfine cellulose, powdered cellulose, pregelatinized starch (e.g., Starch 1500®), calcium phosphate dihydrate, soy polysaccharide (e.g., Emcosoy®), gelatin, silicon dioxide, calcium sulfate, calcium carbonate, magnesium carbonate, magnesium oxide, sorbitol, mannitol, kaolin, polymethacrylates (e.g., Eudragit®), potassium chloride, sodium chloride, and talc. In some embodiments, the ranges for the amount of diluent by weight percent may include about 40% to about 90%, about 50% to about 85%, about 55% to about 80%, about 50% to about 60%, and increments therein.

In embodiments where the composition is compacted into a solid dosage form, e.g., a tablet, a binder can help the ingredients hold together. Binders include, but are not limited to, sugars such as sucrose, lactose, and glucose; corn syrup; soy polysaccharide, gelatin; povidone (e.g., Kollidon®, Plasdone®); Pullulan; cellulose derivatives such as microcrystalline cellulose, hydroxypropylmethyl cellulose (e.g., Methocel®), hydroxypropyl cellulose (e.g., Klucel®), ethylcellulose, hydroxyethyl cellulose, carboxymethylcellulose sodium, and methylcellulose; acrylic and methacrylic acid co-polymers; carbomer (e.g., Carbopol®); polyvinylpolypyrrolidine, polyethylene glycol (Carbowax®); pharmaceutical glaze; alginates such as alginic acid and sodium alginate; gums such as acacia, guar gum, and arabic gums; tragacanth; dextrin and maltodextrin; milk derivatives such as whey; starches such as pregelatinized starch and starch paste; hydrogenated vegetable oil; and magnesium aluminum silicate.

For tablet dosage forms, the composition is subjected to pressure from a punch and dye. Among other purposes, a lubricant can help prevent the composition from sticking to the punch and dye surfaces. A lubricant can also be used in the coating of a coated dosage form. Lubricants include, but are not limited to, magnesium stearate, calcium stearate, zinc stearate, powdered stearic acid, glyceryl monostearate, glyceryl palmitostearate, glyceryl behenate, silica, magnesium silicate, colloidal silicon dioxide, titanium dioxide, sodium benzoate, sodium lauryl sulfate, sodium stearyl fumarate, hydrogenated vegetable oil, talc, polyethylene glycol, and mineral oil. In certain embodiments, the amount of lubricant by weight percent may be less than about 5%, 4%, 3%, 2%, 1.5%, 1%, or 0.5%, or increments therein.

Glidants may improve the flowability of non-compacted solid dosage forms and may improve the accuracy of dosing. Glidants include, but are not limited to, colloidal silicon dioxide, fumed silicon dioxide, silica gel, talc, magnesium trisilicate, magnesium or calcium stearate, powdered cellulose, starch, and tribasic calcium phosphate.

Plasticizers may include both hydrophobic and hydrophilic plasticizers such as, but not limited to, diethyl phthalate, butyl phthalate, diethyl sebacate, dibutyl sebacate, triethyl citrate, acetyltriethyl citrate, acetyltributyl citrate, cronotic acid, propylene glycol, castor oil, triacetin, polyethylene glycol, propylene glycol, glycerin, and sorbitol. Plasticizers are particularly useful for pharmaceutical compositions containing a polymer and in soft capsules and film-coated tablets.

Disintegrants can increase the dissolution rate of a pharmaceutical composition. Disintegrants include, but are not limited to, alginates such as alginic acid and sodium alginate, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), polyvinylpolypyrrolidine (Plasone-XL®), guar gum, magnesium aluminum silicate, methyl cellulose, microcrystalline cellulose, polacrilin potassium, powdered cellulose, starch, pregelatinized starch, sodium starch glycolate (e.g., Explotab®, Primogel®). In some embodiments, the ranges for the amount of disintegrant by weight percent may include about 1% to about 10%, about 1% to about 5%, about 2% to about 3%, and increments therein.

In embodiments where the composition is formulated for a liquid dosage form, the composition may include one or more solvents and/or one or more carriers. Suitable solvents include, but are not limited to, water; alcohols such as ethanol, isopropyl alcohol, propylene glycol, polyethylene glycol, methanol, butanediol, and/or the like; methylene chloride; vegetable oil; and glycerin. Suitable carriers include, but are not limited to, a natural gum, agar, sodium alginate, pectin, methylcellulose, carboxymethylcellulose, saccharose, saccharose with glycerol, mannitol, sorbitol, and polyvinyl alcohol.

The composition may comprise a buffer. Buffers include, but are not limited to, lactic acid, citric acid, acetic acid, sodium lactate, sodium citrate, and sodium acetate.

Any pharmaceutically acceptable colorant can be used to improve appearance or to help identify the composition. Exemplary colorants include D&C Red No. 28, D&C Yellow No. 10, FD&C Blue No. 1, FD&C Red No. 40, FD&C Green #3, FD&C Yellow No. 6, and edible inks. Preferred colors for gelatin capsules include white, medium orange, and light blue.

Flavorings improve palatability and may be particularly useful for chewable tablet or liquid dosage forms. Flavorings include, but are not limited to maltol, vanillin, ethyl vanillin, menthol, citric acid, fumaric acid, ethyl maltol, and tartaric acid. Sweeteners include, but are not limited to, sorbitol, saccharin, sodium saccharin, sucrose, aspartame, fructose, mannitol, and invert sugar.

The composition may also include one or more preservatives and/or stabilizers to improve storability. These include, but are not limited to, alcohol, sodium benzoate, butylated hydroxy toluene (BHT), butylated hydroxyanisole, and ethylenediamine tetraacetic acid.

The composition may further include one or more humectants. These include, but are not limited to, glycerin, lecithin, glycerol, sorbitol, mannitol, sodium hyaluronate, hyaluronic acid, butylene glycol, sodium PCA, allantoin, honey, aloe vera, algae, alpha hydroxy acids, panthenol, carboxylic acid, sodium lactate, ammonium lactate, sodium pyrrolidine, urea, gelatin, and propylene glycol.

The composition may also include one or more antioxidants. These include, but are not limited to, vitamin E, tocopherol acetate, carotenes, BHT, acai oil, alpha lipoic acid, green and white tea extracts, retinol, vitamin C, coenzyme Q10, isoflavones, polyphenols, N-acetylcysteine, curcumin, turmeric, pomegranate extract, rosemary extract, glutathione, selenium, idebenone, lycopene, silymarin, resveratrol, grape seed extract, genistein, pycnogenol, niacinamide, flavonoids, and zinc.

The composition may further include one or more emulsifying agents. These include, but are not limited to, glyceryl stearate citrate, potassium cetyl phosphate, PEG-100, acrylates, xanthan gum, cetaryl alcohol, borax, beeswax, carbomer, lecithin, PEG-20 stearate, propylene glycol, stearyl alcohol NF, polysorbates, laureth-4, and potassium cetyl sulfate.

The composition may also include one or more viscosity enhancing agents. These include, but are not limited to, acrylates, carbopol, polysorbates, squalene, cetyl alcohol, stearyl alcohol, carnauba wax, stearic acid, hydroxyethylcellulose, guar gum, locust bean gum, xanthan gum, gelatin, silica, bentonite, magnesium aluminum silicate, carbomer, and sodium chloride.

The composition may further include one or more emollients. These include, but are not limited to, petrolatum, lanolin, mineral oil, dimethicone, caprylyl methicone, 012-15 alkyl benzoate, dibutyl adipate, dicaprylyl carbonate, isononyl isononanoate, dicapryl ether, dodecane, ethylhexyl palmitate, ethyl macadamate, isohexadecane, capric/caprylic triglyceride, and isoamyl cocoate.

The composition may also include one or more sensory modifiers. These include, but are not limited to, cyclopentasiloxane, dimethicone, other silicones, titanium isopropyl triisostearate, nylon-12, polymethylsilsesquioxane, aluminum starch octenylsuccinate, talc, kaolin, squalene, and glycerine.

The composition may further include one or more cosmetically acceptable carriers. These include, but are not limited to, preservatives, perfumes/fragrances, polymer neutralizers, chelating agents, pH adjusters, and/or the like. Preservatives may include, but are not limited to, tocopherol acetate, propylene glycol, butylene glycol, disodium EDTA, tetrasodium EDTA, iodopropynyl butylcarbamate, phenoxyethanol, diazolidinyl urea, imidazolydyl urea, parabens (e.g., methylparaben, polyparaben, butylparaben, etc.), phenoxyethanol, methylisothiazolinone, ascorbyl palmitate, benzoic acid, and benzyl alcohol. Chelating agents may include, but are not limited to, tetrasodium EDTA, disodium EDTA, and tetrahydroxypropyl ethylenediamine. pH adjusters may include, but are not limited to, citric acid, lactic acid, triethanolamine, sodium gluconate, and magnesium hydroxide.

The composition may also include one or more sunscreens. These include, but are not limited to, zinc oxide, titanium dioxide, oxybenzone, avobenzone, octisalate, octocrylene, homosalate, octinoxate, ensulizole, cinoxate, dioxybenzone, meradimate, padimate O, sulisobenzone, bemotrizinol, diethylaminohydroxybenzoylhexyl benzoate, ethylhexylmethoxy cinnamate, bisoctrizole, benzophenone, 4-aminobenzoic acid, ecamsule, enzacamene, dibenzoylmethane, cinoxate, trolamine salicylate, drometrizole trisiloxane, triethanolamine, retinyl palmitate, bisdisulizole di sodium, amiloxate, polysilicone-15, iscotrizinol, 3-benzylidene camphor, tris-biphenyl triazine, methylisothiazolinone, and ethylhexyl triazone.

Other pharmaceutical additives may include gelling agents such as colloidal clays; thickening agents such as gum tragacanth and sodium alginate; absorption enhancers such as dimethylsulfoxide (DMSO); wetting agents such as lecithin, polysorbates, and laurylsulphates; adsorbents; effervescing agents; surface active agents such as sodium lauryl sulfate, dioctyl sodium sulfosuccinate, triethanolamine, polyoxyethylene sorbitan, poloxalkol, and quaternary ammonium salts; and other miscellaneous excipients such as lactose, mannitol, glucose, fructose, xylose, galactose, sucrose, maltose, xylitol, sorbitol, chloride, sulfate and phosphate salts of potassium, sodium, and magnesium.

### III. Prevention of Genomic Instability

Certain embodiments according to the invention provide compositions and methods for preventing genomic instability and its resulting negative effects. For example, in accordance with certain embodiments, the target compounds may be used to prevent genomic instability connected with at least UV damage, antimicrobial drug resistance, cervical cancer, diseases causing excess mucus production, cosmic radiation, ventilator-induced lung injury, and diagnostic imaging and interventional radiology.

A. UV Damage

Ultraviolet (UV) radiation from the sun and/or tanning beds has the ability to damage DNA in skin cells over time. Repeated exposure and resulting mutations and/or genomic instability can cause the cells to begin growing out of control, possibly resulting in skin cancer. Traditional sunscreens serve as chemical or physical screens to UV rays; however, they do not facilitate an enhanced fidelity of DNA damage repair, and no sunscreen can block 100% of UV rays.

Through combined effort and ingenuity, the inventors have developed a novel preventative composition and method for genomic instability resulting from UV radiation. According to certain embodiments, for example, the composition may comprise a cosmeceutical comprising one or more target compounds. For instance, the composition may be a sunscreen or a cosmetic product (e.g., foundation, powder, etc.) including one or more sunscreens, such as those discussed previously herein. The composition may also include one or more skin care products (e.g., humectants such as aloe vera), steroids, or other additives, such as those discussed previously herein. In such embodiments, for instance, the composition would be a topical composition configured for local administration of the target compound. Because sunscreens are used repeatedly over a long period of time, the composition may include the lowest effective dose of the target compound. For example, in certain embodiments, the target compound dose may be from about 25 mg/m² to about 225 mg/m². In other embodiments, for instance, the target compound dose may be from about 75 mg/m² to about 200 mg/m². For example, according to certain embodiments, the target compound dose may be at least about 25, 50, 75, 100, 125, 150, 175, 180, 185, 190, 195, 200, 205, 210, 215, 220, 221, 222, 223, and 224 mg/m² and/or at most about 225, 200, 175, 150, 125, 100, 75, 50, 45, 40, 35, 30, 29, 28, 27, and 26 mg/m² (e.g., about 100-150 mg/m², about 50-220 mg/m², etc.). Using such a low dose may minimize or eliminate any side effects should the target compound in the composition somehow be introduced systemically, for example, if the composition penetrates more skin layers than intended. Nevertheless, such a dose would still have a positive effect on the skin cells, preventing genomic instability and causing the cells to produce self-protective antioxidants. In this way, the composition may also provide delayed protection due to the induction of MnSOD formation, as previously discussed herein, that can persist from about 4 to about 48 hours after phosphorothioate exposure. In this regard, the composition comprising the target compound and one or more sunscreens may prevent or even reverse damage caused by UV radiation.

B. Antimicrobial Drug Resistance

As a result of misuse and overuse of various antimicrobial medications (particularly antibiotics), microorganisms (e.g., bacteria, viruses, fungi, and parasites) are rapidly mutating into so-called “superbugs” that are able to survive treatment with antimicrobials. As such, the antimicrobials become ineffective and infections persist in the body, thereby increasing the risk of spread to others. Indeed, antimicrobial resistance is recognized by the World Health Organization as a growing threat to global public health, as it threatens the effective prevention and treatment of a rapidly-increasing range of infections caused by bacteria, viruses, fungi, and parasites. As a result, global healthcare costs are increasing due to longer illness duration, additional required testing, and the use of more expensive drugs. Moreover, medical procedures including, but not limited to, organ transplantation, chemotherapy, diabetes management, and major surgery are becoming increasingly high risk.

Through combined effort and ingenuity, the inventors have developed a novel solution to antimicrobial drug resistance by pairing one or more target compounds with one or more antimicrobials in a course of treatment. In this way, and without being bound by theory, it is believed that coupling low non-cytoprotective doses of the target compound with one or more antimicrobials (e.g., antibiotics, antivirals, antifungals, or antiparasitics, as discussed previously herein) may stabilize the genome of the target microbe, thereby reducing or preventing mutation without concomitant cytoprotective effects. In this regard, on a large scale, this treatment may reduce or prevent antimicrobial resistance, and, on an individual scale, this treatment may extend the effect of the antimicrobial after the course of treatment is completed. Additionally, the composition may include one or more additives, such as those discussed previously herein.

Antimicrobial and target compound treatment may be local or systemic depending on the identity and location of the target illness or injury. For example, the composition may be an oral preparation, a topical preparation, an inhalable preparation, or an injectable preparation. Regardless of the preparation, because a course of antimicrobial treatment often lasts days or weeks, a relatively low dose of the target compound should be used. The lowest effective dose of the target compound should be used. In some embodiments, for instance, the target compound dose may be from about 3 mg/m² to about 600 mg/m². In further embodiments, for example, the target compound dose may be from about 75 mg/m² to about 150 mg/m². For example, according to certain embodiments, the target compound dose may be at least 3, 50, 100, 150, 200, 250, 300, 350, 400, 450, 500, 550, 575, 585, 595, 596, 597, 598, and 599 mg/m² and/or at most about 600, 550, 500, 450, 400, 350, 300, 250, 200, 150, 100, 50, 25, 20, 15, 10, 5, and 4 mg/m² (e.g., about 50-200 mg/m², about 3-400 mg/m², etc.). Using a lower dose of target compound with the antimicrobial will permit the beneficial effects of the combination (e.g., reducing or preventing antimicrobial resistance and extending the effect of the antimicrobial), while avoiding any negative effects from prolonged systemic exposure to the target compounds (e.g., protection of pathogenic microbial s).

C. Cervical Cancer

Without regular screenings, cervical cancer frequently goes undetected until it has progressed because it often does not cause any symptoms. The majority of cases of cervical cancer are caused by the human papilloma virus (HPV), which infects cervical cells and may cause them to mutate into cancer cells. For those individuals that have HPV, there are no preventative measures other than regular screenings for early detection of cervical cancer.

Through combined effort and ingenuity, the inventors have developed a novel preventative composition and method for reducing the probability of cervical cancer. According to certain embodiments, for example, the composition may be a topical preparation, e.g., a solution, tablet, cream, gel, suppository, ring, and/or the like for supplying the target compound to the cervix in those individuals with HPV. The application of the target compound to the cells of the cervix may prevent and/or repair mutations in those cells over time. Additionally, the composition may include one or more additives, such as those discussed previously herein.

Because the composition would likely be applied over time, a relatively low dose of the target compound should be used. The lowest effective dose of the target compound should be used. In some embodiments, for instance, the target compound dose may be from about 30 mg/m² to about 600 mg/m². For example, according to certain embodiments, the target compound dose may be at least about 30, 50, 100, 150, 200, 250, 300, 350, 400, 450, 500, 550, 575, 585, 595, 596, 597, 598, and 599 mg/m² and/or at most about 600, 550, 500, 450, 400, 350, 300, 250, 200, 150, 100, 50, 40, 35, 34, 33, 32, and 31 mg/m² (e.g., about 150-400 mg/m², about 30-250 mg/m², etc.).

D. Diseases Causing Excess Mucus Formation

Certain diseases cause excess mucus formation, making it difficult for affected individuals to breathe. Examples of these diseases are cystic fibrosis, chronic bronchitis, pneumonia, and chronic obstructive pulmonary disease (COPD).

Through combined effort and ingenuity, the inventors have developed a novel preventative composition and method for preventing complications arising from excess mucus formation. In particular, the target compounds may reduce the viscosity of mucus in diseases that cause excess mucus formation. According to certain embodiments, for example, the composition may comprise an inhalable, oral, or injectable preparation for supplying one or more target compounds to the lungs to reduce the viscosity of mucus and improve breathing. Additionally, the composition may include one or more additives, such as those discussed previously herein.

Because certain of these diseases that cause excess mucus formation, including, but not limited to, cystic fibrosis, chronic bronchitis, and COPD, are chronic, the composition would need to be administered repeatedly over time. As such, the lowest effective dose of the target compound should be used. In some embodiments, for instance, the target compound dose may be from about 30 mg/m² to about 600 mg/m². For example, according to certain embodiments, the target compound dose may be at least about 30, 50, 100, 150, 200, 250, 300, 350, 400, 450, 500, 550, 575, 585, 595, 596, 597, 598, and 599 mg/m² and/or at most about 600, 550, 500, 450, 400, 350, 300, 250, 200, 150, 100, 50, 40, 35, 34, 33, 32, and 31 mg/m² (e.g., about 150-400 mg/m², about 30-250 mg/m², etc.).

E. Cosmic Radiation Exposure

Cosmic rays are high-energy protons and atomic nuclei originate from the sun (i.e. solar wind) and/or outside the solar system (i.e. galactic cosmic rays) that move through space at nearly the speed of light. These cosmic rays produce cosmic radiation, both in space and when the protons and atomic nuclei interact with components of Earth's atmosphere. Solar flares, which are sudden flashes of increased brightness on the sun that may be accompanied by coronal mass ejections, temporarily increase cosmic radiation, both in space and in Earth's atmosphere. Earth's magnetic field funnels much of the cosmic radiation into radiation belts (i.e. Van Allen radiation belts) that surround Earth, and Earth's atmosphere shields much of the remaining cosmic radiation that makes it through the magnetic field.

Certain professions, however, require increased exposure to cosmic radiation. Astronauts, for example, have the greatest risk. While fears of cosmic radiation exposure have thus far prevented interplanetary missions or any missions that would travel through the Van Allen radiation belts or outside of Earth's magnetic field, there are still significant risks for any missions in low Earth orbit, including to the International Space Station. For example, astronauts have demonstrated increased rates of radiation cataract, lung cancer, breast cancer, ovarian cancer, cognitive and memory changes including Alzheimer's Disease, central nervous system dysfunction, circulatory diseases, and/or the like. While less severe, any profession requiring frequent travel in airplanes (e.g., pilots, flight attendants, frequent flyers for business, etc.) may also be accompanied by risks from cosmic radiation. Indeed, while the baseline level of cosmic radiation for a given flight is typically less than that from an X-ray, solar flares temporarily increase this radiation, and radiation exposure will accumulate in frequent flyers over time.

Through combined effort and ingenuity, the inventors have developed a novel preventative composition and method for preventing genomic instability and its deleterious effects resulting from cosmic radiation. According to certain embodiments, for example, the composition may comprise an oral or injectable preparation for broadly supplying one or more target compounds to the body of individuals at the highest risk of encountering cumulative cosmic radiation. Additionally, the composition may include one or more additives, such as those discussed previously herein.

Because the effected individuals would be repeatedly exposed to cosmic radiation over a long period of time (e.g., days, weeks, months, or even years), the composition would need to be administered repeatedly over time, not to exceed 20-30 doses to avoid cumulative toxicity. As such, the lowest effective dose of the target compound should be used. In some embodiments, for instance, the target compound dose may be from about 30 mg/m² to about 600 mg/m². For example, according to certain embodiments, the target compound dose may be at least about 30, 50, 100, 150, 200, 250, 300, 350, 400, 450, 500, 550, 575, 585, 595, 596, 597, 598, and 599 mg/m² and/or at most about 600, 550, 500, 450, 400, 350, 300, 250, 200, 150, 100, 50, 40, 35, 34, 33, 32, and 31 mg/m² (e.g., about 150-400 mg/m², about 30-250 mg/m², etc.).

F. Ventilator-Induced Lung Injury

Ventilators provide mechanical ventilation to the body by pumping breathable air into and out of the lungs and are used when a patient cannot breathe adequately on their own. For example, ventilators are used for comas, pneumonia, COPD, stroke, brain injuries, collapsed lungs, drug overdoses, amyotrophic lateral sclerosis (ALS), Guillain-Barré syndrome, lung infection, myasthenia gravis, polio, premature lung development, upper spinal cord injuries, and/or the like. Ventilators, however, can cause lung injury resulting from reactive oxygen species production and oxidative stress, which, in more severe cases, may lead to increased mortality. These risks have never been more evident than with the COVID-19 pandemic. Indeed, as a result of the COVID-19 pandemic, ventilator use and demand has skyrocketed, as have cases of ventilator-induced lung injury and mortality.

Through combined effort and ingenuity, the inventors have developed a novel preventative composition and method for reducing and/or preventing ventilator-induced lung injury and mortality. According to certain embodiments, for example, the composition may comprise an inhalable, oral, or injectable preparation for supplying one or more target compounds to the lungs to protect lung tissue from the reactive oxygen species and oxidative stress that occur as a result of ventilator use. In some embodiments, for instance, the composition may be administered to a subject prior to being put on a ventilator, currently on a ventilator, or recently removed from a ventilator. Additionally, the composition may include one or more additives, such as those discussed previously herein.

Because ventilators are often used over an extended period of time, the composition would need to be administered repeatedly over time. As such, the lowest effective dose of the target compound should be used. In some embodiments, for instance, the target compound dose may be from about 30 mg/m² to about 600 mg/m². For example, according to certain embodiments, the target compound dose may be at least about 30, 50, 100, 150, 200, 250, 300, 350, 400, 450, 500, 550, 575, 585, 595, 596, 597, 598, and 599 mg/m² and/or at most about 600, 550, 500, 450, 400, 350, 300, 250, 200, 150, 100, 50, 40, 35, 34, 33, 32, and 31 mg/m² (e.g., about 150-400 mg/m², about 30-250 mg/m², etc.).

G. Diagnostic Imaging and Interventional Radiology

Diagnostic imaging and interventional radiology have enormous potential in diagnosing and treating various conditions. Examples of diagnostic imaging include, but are not limited to, nuclear medicine (e.g., bone scan, thyroid scan, thallium cardiac stress test, etc.), computed tomography (CT), CT angiography, fluoroscopy, magnetic resonance imaging (MRI), magnetic resonance angiography (MRA), mammography, x-rays, positron emission tomography (PET), PET-CT, ultrasound, and/or the like. Examples of interventional radiology procedures include angiography or angioplasty and stent placement, embolization to control bleeding, tumor embolization, tumor ablation, vertebroplasty, kyphoplasty, guided needle biopsies, guided breast biopsies, uterine artery embolization, feeding tube placement, venous access catheter placement, and/or the like. Doctors and patients, however, may be hesitant to use some of these procedures due to their risk of increased radiation exposure and resulting genomic instability and mutations, particularly if many procedures are needed over a period of time.

Through combined effort and ingenuity, the inventors have developed a novel preventative composition and method for preventing genomic instability resulting from various diagnostic imaging and interventional radiology procedures. According to certain embodiments, for example, the composition may comprise an oral or injectable preparation for supplying one or more target compounds to region of the body being exposed to diagnostic imaging or interventional radiology procedures. Additionally, the composition may include one or more additives, such as those discussed previously herein.

Because the goal in administering the composition would be to prevent genomic instability resulting from these procedures, and there is a possibility of repeated procedures, the lowest effective dose of the target compound should be used. In some embodiments, for instance, the target compound dose may be from about 10 mg/m² to about 600 mg/m². In further embodiments, for example, the target compound dose may be from about 30 mg/m² to about 200 mg/m². For example, according to certain embodiments, the target compound dose may be at least about 30, 50, 100, 150, 200, 250, 300, 350, 400, 450, 500, 550, 575, 585, 595, 596, 597, 598, and 599 mg/m² and/or at most about 600, 550, 500, 450, 400, 350, 300, 250, 200, 150, 100, 50, 40, 35, 34, 33, 32, and 31 mg/m² (e.g., about 150-400 mg/m², about 30-250 mg/m², etc.).

### IV. Cytoprotection

Certain embodiments according to the invention provide compositions and methods for protecting against cell death. For example, in accordance with certain embodiments, one or more target compounds may be used to protect against cell death resulting in at least radiation dermatitis, chemotherapy- and radiation therapy-induced hair loss, alopecia areata, acute radiation syndrome (ARS), radiation-induced fibrosis, chemotherapy associated peripheral neuropathy, radiation therapy- and chemotherapy-induced infertility, and radiation therapy-induced organ damage.

A. Radiation Dermatitis

Radiation dermatitis is a common side effect of radiotherapy, which may damage or even kill skin cells as it also damages and kills cancer cells. Symptoms may include redness, skin peeling, and ulceration, and they typically begin to resolve after treatment ends. However, in some cases symptoms may appear years after radiotherapy is complete.

Through combined effort and ingenuity, the inventors have developed compositions and methods for protecting skin cells from radiation in order to reduce radiation dermatitis or prevent its development entirely. According to certain embodiments, the composition may comprise one or more target compounds in a local topical preparation. The composition may be a local topical preparation in order to prevent the higher dose of the target compound needed to protect from radiation dermatitis from becoming systemic. The composition may also include one or more skin care products (e.g., humectants such as aloe vera), steroids, or other additives, such as those discussed previously herein (e.g., beta-glucan, prostaglandins, etc.).

Because the composition would be used to protect against a high dose of radiation, a relatively high dose of the target compound may be used. Indeed, the highest dose possible that can be tolerated may be administered. In some embodiments, for instance, the target compound dose may be from about 200 mg/m² to about 2200 mg/m² may be used. In further embodiments, for example, a dose from about 600 mg/m² to about 1200 mg/m² may be used. In this regard, for instance, the dose used may be from at least about any of the following: 200, 300, 400, 500, 600, 700, 800, 900, 1000, 1100, 1200, 1300, 1400, 1500, 1600, 1700, 1800, 1900, 2000, 2100, 2150, 2160, 2170, 2180, 2190, 2195, 2196, 2197, 2198, and 2199 mg/m² and/or at most about 2200, 2100, 2000, 1900, 1800, 1700, 1600, 1500, 1400, 1300, 1200, 1100, 1000, 900, 800, 700, 600, 500, 400, 300, 250, 240, 230, 220, 210, 205, 204, 203, 202, and 201 mg/m² (e.g., about 700-1000 mg/m², about 200-900 mg/m², etc.). In cases in which radiation exposure will be repeated over an extended period of time, then dosages at the lower end of the above range should be used to avoid cumulative toxicity. For example, in such cases a dose from about 200 mg/m² to about 600 mg/m², or even 200 mg/m² to about 350 mg/m², may be used. This topical preparation may be applied to the skin within minutes (e.g., about 15-30 minutes) prior to radiation exposure in order to prevent the target compound from becoming systemic should the composition penetrate deeper into the skin. In further embodiments, the composition may include an accelerator (e.g., DMSO) such that the composition may be applied five minutes or less before exposure.

B. Radiation Therapy- and Chemotherapy-Induced Hair Loss

As discussed previously herein, chemotherapy targets rapidly-dividing cells, as cancer cells tend to divide quite rapidly. Unfortunately, hair follicles also have rapidly-dividing cells, and chemotherapy does not distinguish between rapidly-dividing cancerous and healthy cells. Similarly, hair follicles, like cancer cells, are very sensitive to radiation, so radiation therapy may have negative effects on hair follicles while killing cancer cells. As a result, hair loss is a common side effect of cancer treatment.

Through combined effort and ingenuity, the inventors have developed compositions and methods for protecting hair follicles from radiation and/or chemotherapy in order to reduce hair loss or prevent its development entirely. According to certain embodiments, the composition may comprise one or more target compounds in a local topical preparation, e.g., a leave-in conditioner, serum, cream, mask, and/or the like. The topical preparation may include one or more steroids and/or other additives, including, but not limited to, glycerol, propylene glycol, panthenol, erythritol, sodium PCA, hyaluronic acid, sorbitol, fructose, fatty alcohols, polyquaternium polymers, cationic surfactants (e.g., cetrimonium chloride, dicetyldimonium chloride, etc.), proteins, hydrolyzed proteins, amino acids, plant oils, mineral oil, silicones (e.g., dimethicone, amodimethicone, cyclomethicone, dimethiconol, etc.), esters (e.g., glyceryl stearate, isopropyl palmitate, etc.), fatty acids (e.g., coconut fatty acid, stearic acid, lauric acid, etc.), behentrimonium chloride, stearalkonium chloride, amine oxides, cetrimonium chloride, citric acid, ascorbates, citrus extracts, glycerin, ethylhexyl methoxycinnamate, benzophenone, polyamide-2, salicylates, PABA, dimethylparamidopropyl laurdimonium tosylate, and/or the like. The composition may include one or more additional additives, such as those discussed previously herein. The composition may be a local topical preparation in order to prevent the higher dose of the target compound needed to protect against hair loss from becoming systemic.

Because the composition would be used to protect against a high dose of radiation, a relatively high dose of the target compound may be used. Indeed, the highest dose possible that can be tolerated may be administered. In some embodiments, for instance, the target compound dose may be from about 200 mg/m² to about 2200 mg/m² may be used. In further embodiments, for example, a dose from about 600 mg/m² to about 1200 mg/m² may be used. In this regard, for instance, the dose used may be from at least about any of the following: 200, 300, 400, 500, 600, 700, 800, 900, 1000, 1100, 1200, 1300, 1400, 1500, 1600, 1700, 1800, 1900, 2000, 2100, 2150, 2160, 2170, 2180, 2190, 2195, 2196, 2197, 2198, and 2199 mg/m² and/or at most about 2200, 2100, 2000, 1900, 1800, 1700, 1600, 1500, 1400, 1300, 1200, 1100, 1000, 900, 800, 700, 600, 500, 400, 300, 250, 240, 230, 220, 210, 205, 204, 203, 202, and 201 mg/m² (e.g., about 700-1000 mg/m², about 200-900 mg/m², etc.). In cases in which radiation exposure will be repeated over an extended period of time, then dosages at the lower end of the above range should be used to avoid cumulative toxicity. For example, in such cases a dose from about 200 mg/m² to about 600 mg/m², or even 200 mg/m² to about 350 mg/m², may be used. This topical preparation may be applied to the skin within minutes (e.g., about 15-30 minutes) prior to radiation exposure in order to prevent the target compound from becoming systemic should the composition penetrate deeper into the skin. In further embodiments, the composition may include an accelerator (e.g., DMSO) such that the composition may be applied five minutes or less before exposure.

C. Alopecia Areata

Alopecia areata is an autoimmune disorder that typically results in unpredictable, patchy hair loss when the immune system attacks hair follicles.

Through combined effort and ingenuity, the inventors have developed compositions and methods for protecting hair follicles from the immune system in order to reduce hair loss or prevent its development entirely. According to certain embodiments, the composition may comprise one or more target compounds in a local topical preparation, e.g., a leave-in conditioner, serum, cream, mask, and/or the like. The topical preparation may include one or more steroids and/or other additives, including, but not limited to, glycerol, propylene glycol, panthenol, erythritol, sodium PCA, hyaluronic acid, sorbitol, fructose, fatty alcohols, polyquaternium polymers, cationic surfactants (e.g., cetrimonium chloride, dicetyldimonium chloride, etc.), proteins, hydrolyzed proteins, amino acids, plant oils, mineral oil, silicones (e.g., dimethicone, amodimethicone, cyclomethicone, dimethiconol, etc.), esters (e.g., glyceryl stearate, isopropyl palmitate, etc.), fatty acids (e.g., coconut fatty acid, stearic acid, lauric acid, etc.), behentrimonium chloride, stearalkonium chloride, amine oxides, cetrimonium chloride, citric acid, ascorbates, citrus extracts, glycerin, ethylhexyl methoxycinnamate, benzophenone, polyamide-2, salicylates, PABA, dimethylparamidopropyl laurdimonium tosylate, and/or the like.

Because the composition would be used as a cytoprotectant, a relatively high dose of the target compound may be used. Indeed, the highest dose possible that can be tolerated may be administered. In some embodiments, for instance, the target compound dose may be from about 200 mg/m² to about 2200 mg/m² may be used. In further embodiments, for example, a dose from about 600 mg/m² to about 1200 mg/m² may be used. In this regard, for instance, the dose used may be from at least about any of the following: 200, 300, 400, 500, 600, 700, 800, 900, 1000, 1100, 1200, 1300, 1400, 1500, 1600, 1700, 1800, 1900, 2000, 2100, 2150, 2160, 2170, 2180, 2190, 2195, 2196, 2197, 2198, and 2199 mg/m² and/or at most about 2200, 2100, 2000, 1900, 1800, 1700, 1600, 1500, 1400, 1300, 1200, 1100, 1000, 900, 800, 700, 600, 500, 400, 300, 250, 240, 230, 220, 210, 205, 204, 203, 202, and 201 mg/m² (e.g., about 700-1000 mg/m², about 200-900 mg/m², etc.). Because treatment may require multiple applications of the composition, dosages at the lower end of the above range may be used to avoid cumulative toxicity. For example, in such cases a dose from about 200 mg/m² to about 600 mg/m² may be used.

D. Acute Radiation Syndrome (ARS)

Acute radiation syndrome (ARS) (also known as radiation toxicity, radiation poisoning, or radiation sickness) is an acute illness caused by irradiation of most or all of the body by a high dose of penetrating radiation in a very short period of time. Sources of the radiation can include nuclear reactors, cyclotrons, atomic bombs, certain devices used in cancer therapy, and/or the like. Symptoms may include nausea, vomiting, loss of appetite, abdominal pain, hematopoietic syndrome, dizziness, headache, decreased level of consciousness, fatigue, fever, skin reddening, seizures, lung inflammation and scarring, internal bleeding, burns, tremor, ataxia, lethargy, leukopenia, weakness, purpura, infections, alopecia, diarrhea, hypotension, electrolyte disturbance, shock, itching, blistering, ulceration, damaged sebaceous and sweat glands, atrophy, fibrosis, decreased or increased skin pigmentation, necrosis, moist desquamation, dermal vascular system collapse, and/or the like. ARS is of particular concern for first-responders during any kind of nuclear event.

Through combined effort and ingenuity, the inventors have developed compositions and methods for protecting the body from radiation in order to reduce the severity of ARS or prevent its development entirely. Indeed, in some embodiments, the composition may protect against gastrointestinal toxicity, hematopoietic toxicity, central nervous system toxicity, skin toxicity, or any combination thereof. According to certain embodiments, the composition may comprise one or more target compounds in an oral, injectable, inhalable, or topical preparation. Additionally, the composition may include one or more additives (e.g., one or more steroids), such as those discussed previously herein.

Because the composition would be used to protect against a high dose of radiation, a relatively high dose of the target compound may be used. Indeed, the highest dose possible that can be tolerated may be administered. In some embodiments, for instance, the target compound dose may be from about 200 mg/m² to about 2200 mg/m² may be used. In further embodiments, for example, a dose from about 600 mg/m² to about 1200 mg/m² may be used. In this regard, for instance, the dose used may be from at least about any of the following: 200, 300, 400, 500, 600, 700, 800, 900, 1000, 1100, 1200, 1300, 1400, 1500, 1600, 1700, 1800, 1900, 2000, 2100, 2150, 2160, 2170, 2180, 2190, 2195, 2196, 2197, 2198, and 2199 mg/m² and/or at most about 2200, 2100, 2000, 1900, 1800, 1700, 1600, 1500, 1400, 1300, 1200, 1100, 1000, 900, 800, 700, 600, 500, 400, 300, 250, 240, 230, 220, 210, 205, 204, 203, 202, and 201 mg/m² (e.g., about 700-1000 mg/m², about 200-900 mg/m², etc.). The composition may be administered within minutes (e.g., about 15-30 minutes) prior to radiation exposure for maximum effectiveness. In further embodiments, the composition may include an accelerator (e.g., DMSO) such that the composition may be applied five minutes or less before exposure. In certain embodiments, the composition may further include one or more additional drugs (e.g., beta-glucan) that boost the radioprotective effects of the target compounds, thereby reducing the required dosage of the target compounds to achieve the desired cytoprotective effects.

E. Radiation-Induced Fibrosis

While radiation therapy kills cancer cells, it also may negatively affect healthy tissues within the radiation field. One side effect that may develop is radiation-induced fibrosis, which can occur in the skin and subcutaneous tissue, lungs, gastrointestinal and genitourinary tracts, and any other organs within the radiation field. Specifically, the radiation therapy may trigger inflammation and cause fibroblasts to transform into myofibroblasts. Myofibroblasts are known to rapidly and excessively proliferate and also to produce excess collagen and other extracellular matrix components. The resulting fibrosis may cause functional and cosmetic impairments including skin induration and thickening, muscle shortening and atrophy, limited joint mobility, lymphedema, mucosal fibrosis, ulceration, fistula, hollow organ stenosis, pain, trismus, xerostomia, decreased vocal quality, osteoradionecrosis, dysphagia, aspiration, cervical plexopathy, brachial plexopathy, interstitial fibrosis, dyspnea, oxygen requirement, urinary urgency, increased urinary frequency, diarrhea, loss of reproductive function, dyspareunia, other physical trauma, gradual ischemia, and/or the like.

Through combined effort and ingenuity, the inventors have developed a novel preventative composition and method for preventing radiation-induced fibrosis. According to certain embodiments, for example, the composition may comprise an inhalable, oral, topical, or injectable preparation for supplying one or more target compounds to one or more regions in the body within the radiation field to protect those tissues at risk for developing radiation-induced fibrosis. Additionally, the composition may include one or more additives, such as those discussed previously herein.

Because the composition would be used to protect against a high dose of radiation, a relatively high dose of the target compound may be used. Indeed, the highest dose possible that can be tolerated may be administered. In some embodiments, for instance, the target compound dose may be from about 200 mg/m² to about 2200 mg/m² may be used. In further embodiments, for example, a dose from about 600 mg/m² to about 1200 mg/m² may be used. In this regard, for instance, the dose used may be from at least about any of the following: 200, 300, 400, 500, 600, 700, 800, 900, 1000, 1100, 1200, 1300, 1400, 1500, 1600, 1700, 1800, 1900, 2000, 2100, 2150, 2160, 2170, 2180, 2190, 2195, 2196, 2197, 2198, and 2199 mg/m² and/or at most about 2200, 2100, 2000, 1900, 1800, 1700, 1600, 1500, 1400, 1300, 1200, 1100, 1000, 900, 800, 700, 600, 500, 400, 300, 250, 240, 230, 220, 210, 205, 204, 203, 202, and 201 mg/m² (e.g., about 700-1000 mg/m², about 200-900 mg/m², etc.). In cases in which radiation exposure will be repeated over an extended period of time, then dosages at the lower end of the above range should be used to avoid cumulative toxicity. For example, in such cases a dose from about 200 mg/m² to about 600 mg/m² may be used.

F. Chemotherapy Associated Peripheral Neuropathy

As with radiation therapy, chemotherapy may negatively affect healthy cells in addition to cancer cells. One potential negative effect of chemotherapy is damage to peripheral nerves, resulting in peripheral neuropathy. The peripheral nerves carry sensation, control arm and leg movements, and control the bladder and bowel, and symptoms of peripheral neuropathy may include tingling, pain, decreased sensation, increased sensitivity to touch, temperature, pressure, and/or pain, muscle weakness, difficulty with fine motor skills, and/or the like.

Through combined effort and ingenuity, the inventors have developed a novel preventative composition and method for preventing chemotherapy associated peripheral neuropathy. According to certain embodiments, for example, the composition may comprise an oral or injectable preparation for supplying one or more target compounds to the peripheral nerves to protect the nerve cells from the chemotherapy drugs. Additionally, the composition may include one or more additives, such as those discussed previously herein.

Because the composition would be used to protect against a high dose of radiation, a relatively high dose of the target compound may be used. Indeed, the highest dose possible that can be tolerated may be administered. In some embodiments, for instance, the target compound dose may be from about 200 mg/m² to about 2200 mg/m² may be used. In further embodiments, for example, a dose from about 600 mg/m² to about 1200 mg/m² may be used. In this regard, for instance, the dose used may be from at least about any of the following: 200, 300, 400, 500, 600, 700, 800, 900, 1000, 1100, 1200, 1300, 1400, 1500, 1600, 1700, 1800, 1900, 2000, 2100, 2150, 2160, 2170, 2180, 2190, 2195, 2196, 2197, 2198, and 2199 mg/m² and/or at most about 2200, 2100, 2000, 1900, 1800, 1700, 1600, 1500, 1400, 1300, 1200, 1100, 1000, 900, 800, 700, 600, 500, 400, 300, 250, 240, 230, 220, 210, 205, 204, 203, 202, and 201 mg/m² (e.g., about 700-1000 mg/m², about 200-900 mg/m², etc.). In cases in which radiation exposure will be repeated over an extended period of time, then dosages at the lower end of the above range should be used to avoid cumulative toxicity. For example, in such cases a dose from about 200 mg/m² to about 600 mg/m², or even 200 mg/m² to about 350 mg/m², may be used.

G. Radiation Therapy- and Chemotherapy-Induced Infertility

Radiation directed at the pelvis and/or reproductive organs may cause infertility in cancer patients. Similarly, because egg and sperm cells divide rapidly and because chemotherapy targets rapidly-dividing cells, chemotherapy may also cause infertility in cancer patients. There are currently no means for preserving fertility without retrieving and preserving egg or sperm cells prior to treatment. However, as understood by one of ordinary skill in the art, the differences between sperm and egg cells may lead to different courses of treatment in males and females. For example, while mature sperm cells are very radiation resistant, the process of spermatogenesis and the sperm stem cells used therein are what need to be protected from mutation. On the other hand, all of a woman's egg cells are present at birth, so protection against mutation and cell death would both be beneficial.

Through combined effort and ingenuity, the inventors have developed a novel preventative composition and method for reducing the likelihood of infertility in cancer patients. Similarly, the composition may be used to prevent mutagenesis in those sperm and/or egg cells that survive radiation and/or chemotherapy. According to certain embodiments, for example, the composition may comprise an oral or injectable preparation for supplying one or more target compounds to the egg or sperm cells in the reproductive organs. Additionally, the composition may include one or more additives, such as those discussed previously herein.

The dosage of the composition will depend on whether the goal is to protect the egg and/or stem cells from genomic instability/mutagenesis or cell death. For example, to protect against sterility in females, cytoprotective doses are required. In that scenario, the highest dose possible that can be tolerated may be administered. In some embodiments, for instance, the target compound dose may be from about 200 mg/m² to about 2200 mg/m² may be used. In further embodiments, for example, a dose from about 600 mg/m² to about 1200 mg/m² may be used. In this regard, for instance, the dose used may be from at least about any of the following: 200, 300, 400, 500, 600, 700, 800, 900, 1000, 1100, 1200, 1300, 1400, 1500, 1600, 1700, 1800, 1900, 2000, 2100, 2150, 2160, 2170, 2180, 2190, 2195, 2196, 2197, 2198, and 2199 mg/m² and/or at most about 2200, 2100, 2000, 1900, 1800, 1700, 1600, 1500, 1400, 1300, 1200, 1100, 1000, 900, 800, 700, 600, 500, 400, 300, 250, 240, 230, 220, 210, 205, 204, 203, 202, and 201 mg/m² (e.g., about 700-1000 mg/m², about 200-900 mg/m², etc.). In cases in which radiation or chemotherapy exposure will be repeated over an extended period of time, then dosages at the lower end of the above range should be used to avoid cumulative toxicity. For example, in such cases a dose from about 200 mg/m² to about 600 mg/m², or even 200 mg/m² to about 350 mg/m², may be used.

To protect against genomic instability/mutagenesis in males or females, the lowest effective dose of the target compound should be used. In some embodiments, for instance, the target compound dose may be from about 10 mg/m² to about 600 mg/m². In further embodiments, for example, the target compound dose may be from about 30 mg/m² to about 200 mg/m². For example, according to certain embodiments, the target compound dose may be at least about 30, 50, 100, 150, 200, 250, 300, 350, 400, 450, 500, 550, 575, 585, 595, 596, 597, 598, and 599 mg/m² and/or at most about 600, 550, 500, 450, 400, 350, 300, 250, 200, 150, 100, 50, 40, 35, 34, 33, 32, and 31 mg/m² (e.g., about 150-400 mg/m², about 30-250 mg/m², etc.).

H. Radiation Therapy-Induced Organ Damage

Radiation directed at internal organs may damage those organs in addition to cancer cells. Currently, there are no effective means of protecting certain organs, namely the liver, kidney, spleen, pancreas, lung, heart, skin, reproductive organs, prostate, colon, small intestine, and rectum, from radiation damage.

Through combined effort and ingenuity, the inventors have developed a novel preventative composition and method for preventing radiation therapy-induced damage in the liver, kidneys, and spleen. According to certain embodiments, for example, the composition may comprise an oral or injectable preparation for supplying one or more target compounds to the liver, kidneys, spleen, pancreas, lung, heart, skin, reproductive organs, prostate, colon, small intestine, and/or rectum. Without being bound by theory, the liver, kidneys, and spleen are particularly amenable to treatment by the target compounds because the liver, kidneys, spleen, pancreas, lung, heart, skin, reproductive organs, prostate, colon, small intestine, and rectum take up the target compounds better than tumor cells, thereby maximizing the benefits to these organs while avoiding protecting tumor metastases.

Because the composition would be used to protect against a high dose of radiation, a relatively high dose of the target compound may be used. Indeed, the highest dose possible that can be tolerated may be administered. In some embodiments, for instance, the target compound dose may be from about 200 mg/m² to about 2200 mg/m² may be used. In further embodiments, for example, a dose from about 600 mg/m² to about 1200 mg/m² may be used. In this regard, for instance, the dose used may be from at least about any of the following: 200, 300, 400, 500, 600, 700, 800, 900, 1000, 1100, 1200, 1300, 1400, 1500, 1600, 1700, 1800, 1900, 2000, 2100, 2150, 2160, 2170, 2180, 2190, 2195, 2196, 2197, 2198, and 2199 mg/m² and/or at most about 2200, 2100, 2000, 1900, 1800, 1700, 1600, 1500, 1400, 1300, 1200, 1100, 1000, 900, 800, 700, 600, 500, 400, 300, 250, 240, 230, 220, 210, 205, 204, 203, 202, and 201 mg/m² (e.g., about 700-1000 mg/m², about 200-900 mg/m², etc.). In cases in which radiation exposure will be repeated over an extended period of time, then dosages at the lower end of the above range should be used to avoid cumulative toxicity. For example, in such cases a dose from about 200 mg/m² to about 600 mg/m², or even 200 mg/m² to about 350 mg/m², may be used. Additionally, the composition may include one or more additives, such as those discussed previously herein.

### Non-Limiting Exemplary Embodiments

Having described various aspects and embodiments of the invention herein, further specific embodiments of the invention include those set forth in the following paragraphs.

Certain embodiments according to the invention provide compositions and methods for preventing and reversing ultraviolet (UV) damage.

In one aspect, a composition for preventing and reversing ultraviolet (UV) radiation damage is provided. In accordance with certain embodiments, the composition includes a target compound and a sunscreen. In some embodiments, the target compound comprises a phosphorothioate compound having one or more disulfide, mixed disulfide, or free thiol groups and a polyamine backbone with positively charged amine groups or an analog, derivative, prodrug, or salt thereof. In certain embodiments, the composition reduces or prevents genomic instability resulting from UV radiation. In further embodiments, the composition induces antioxidant production in skin cells to reverse UV radiation damage.

In accordance with certain embodiments, the target compound comprises at least one of S-2-(3-aminopropylamino)ethyl phosphorothioic acid (amifostine, WR-2721), 2-[(aminopropyl)amino]ethanethiol (amifostine free thiol form, WR-1065), [2-[(aminopropyl) amino] ethanethiol] N,N,′-dithiodi-2,1-(ethanediyl) bis-1,3-propanediamine (amifostine disulfide form, WR-33278), S-[2-(3-methylaminopropyl)aminoethyl]phosphorothioate acid (phosphonol, WR-3689), 2-[3-(methyl amino) propylamino] ethanethiol (WR-255591), S-3-(3-methylaminopropylamino) propyl phosphorothioic acid (WR-151327), 3-(3-methyl aminopropyl amino) propanethiol dihydrochloride (WR-151326), or a mixture thereof. In further embodiments, the composition further comprises a steroid.

In accordance with certain embodiments, the sunscreen comprises at least one of zinc oxide, titanium dioxide, oxybenzone, avobenzone, octisalate, octocrylene, homosalate, octinoxate, ensulizole, cinoxate, dioxybenzone, meradimate, padimate O, sulisobenzone, bemotrizinol, diethylaminohydroxybenzoylhexyl benzoate, ethylhexylmethoxy cinnamate, bisoctrizole, benzophenone, 4-aminobenzoic acid, ecamsule, enzacamene, dibenzoylmethane, cinoxate, trolamine salicylate, drometrizole trisiloxane, triethanolamine, retinyl palmitate, bisdisulizole disodium, amiloxate, poly silicone-15, iscotrizinol, 3-benzylidene camphor, tris-biphenyl triazine, methylisothiazolinone, ethylhexyl triazone, or a mixture thereof.

In accordance with certain embodiments, the composition includes the target composition at a dosage from about 25 mg/m² to about 225 mg/m². In other embodiments, the composition includes the target composition at a dosage from about 75 mg/m² to about 200 mg/m².

In accordance with certain embodiments, the composition comprises a sunscreen lotion or a cosmetic product. In some embodiments, the composition comprises a topical preparation.

In another aspect, a method of preventing and reversing UV radiation damage is provided. In accordance with certain embodiments, the method includes applying a composition to skin. In some embodiments, the composition comprises a target compound and a sunscreen. In further embodiments, the target compound comprises a phosphorothioate compound having one or more disulfide, mixed disulfide, or free thiol groups and a polyamine backbone with positively charged amine groups or an analog, derivative, prodrug, or salt thereof.

In accordance with certain embodiments, applying the composition to the skin comprises applying the composition to the skin up to 12 hours before UV exposure and/or up to 24 hours after UV exposure.

In certain embodiments, the composition reduces or prevents genomic instability resulting from UV radiation. In further embodiments, the composition induces antioxidant production in skin cells to reverse UV radiation damage.

In accordance with certain embodiments, the target compound comprises at least one of S-2-(3-aminopropylamino)ethyl phosphorothioic acid (amifostine, WR-2721), 2-[(aminopropyl)amino]ethanethiol (amifostine free thiol form, WR-1065), [2-[(aminopropyl) amino] ethanethiol] N,N,′-dithiodi-2,1-(ethanediyl) bis-1,3-propanediamine (amifostine disulfide form, WR-33278), S-[2-(3-methylaminopropyl)aminoethyl]phosphorothioate acid (phosphonol, WR-3689), 2-[3-(methyl amino) propylamino] ethanethiol (WR-255591), S-3-(3-methylaminopropylamino) propyl phosphorothioic acid (WR-151327), 3-(3-methyl aminopropyl amino) propanethiol dihydrochloride (WR-151326), or a mixture thereof. In further embodiments, the composition further comprises a steroid.

In accordance with certain embodiments, the sunscreen comprises at least one of zinc oxide, titanium dioxide, oxybenzone, avobenzone, octisalate, octocrylene, homosalate, octinoxate, ensulizole, cinoxate, dioxybenzone, meradimate, padimate 0, sulisobenzone, bemotrizinol, diethylaminohydroxybenzoylhexyl benzoate, ethylhexylmethoxy cinnamate, bisoctrizole, benzophenone, 4-aminobenzoic acid, ecamsule, enzacamene, dibenzoylmethane, cinoxate, trolamine salicylate, drometrizole trisiloxane, triethanolamine, retinyl palmitate, bisdisulizole disodium, amiloxate, poly silicone-15, iscotrizinol, 3-benzylidene camphor, tris-biphenyl triazine, methylisothiazolinone, ethylhexyl triazone, or a mixture thereof.

In accordance with certain embodiments, the composition includes the target composition at a dosage from about 25 mg/m² to about 225 mg/m². In other embodiments, the composition includes the target composition at a dosage from about 75 mg/m² to about 200 mg/m².

In accordance with certain embodiments, the composition comprises a sunscreen lotion or a cosmetic product.

Modifications of the invention set forth herein will come to mind to one skilled in the art to which the invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1-153. (canceled)

154. A composition for preventing genomic instability from a diagnostic imaging or interventional radiology procedure, the composition comprising a target compound, the target compound comprising a phosphorothioate compound having one or more disulfide, mixed disulfide, or free thiol groups and a polyamine backbone with positively charged amine groups or an analog, derivative, prodrug, or salt thereof.

155. The composition according to claim 154, wherein the target compound comprises at least one of S-2-(3-aminopropylamino)ethyl phosphorothioic acid (amifostine, WR-2721), 2-[(aminopropyl)amino]ethanethiol (amifostine free thiol form, WR-1065), [2-[(aminopropyl) amino] ethanethiol] N,N,′-dithiodi-2,1-(ethanediyl) bis-1,3-propanediamine (amifostine disulfide form, WR-33278), S-[2-(3-methylaminopropyl)aminoethyl]phosphorothioate acid (phosphonol, WR-3689), 2-[3-(methylamino) propylamino] ethanethiol (WR-255591), S-3-(3-methylaminopropylamino) propyl phosphorothioic acid (WR-151327), 3-(3-methylaminopropylamino) propanethiol dihydrochloride (WR-151326), or a mixture thereof.

156. The composition according to claim 154, wherein the diagnostic imaging procedure comprises nuclear medicine imaging, computed tomography (CT), CT angiography, fluoroscopy, magnetic resonance imaging (MRI), magnetic resonance angiography (MRA), mammography, x-ray, positron emission tomography (PET), PET-CT, ultrasound, or a combination thereof.

157. The composition according to claim 154, wherein the interventional radiology procedure comprises stent placement via angiography or angioplasty, embolization to control bleeding, tumor embolization, tumor ablation, vertebroplasty, kyphoplasty, guided needle biopsy, guided breast biopsy, uterine artery embolization, feeding tube placement, venous access catheter placement, or a combination thereof.

158. The composition according to claim 154, wherein the composition includes the target composition at a dosage from about 10 mg/m² to about 600 mg/m².

159. The composition according to claim 154, wherein the composition is configured for a single systemic administration.

160. The composition according to claim 154, wherein the composition is configured for repeated systemic administration.

161. The composition according to claim 154, wherein the composition comprises an oral preparation or an injectable preparation.

162. A method of preventing genomic instability from a diagnostic imaging or interventional radiology procedure, the method comprising administering a composition to a subject in need thereof, the composition comprising a target compound, the target compound comprising a phosphorothioate compound having one or more disulfide, mixed disulfide, or free thiol groups and a polyamine backbone with positively charged amine groups or an analog, derivative, prodrug, or salt thereof.

163. The method according to claim 162, wherein administering the composition comprises systemically administering the composition in a single dose.

164. The method according to claim 162, wherein administering the composition comprises systemically administering the composition over 20-30 doses.

165. The method according to claim 162, wherein the diagnostic imaging procedure comprises nuclear medicine imaging, computed tomography (CT), CT angiography, fluoroscopy, magnetic resonance imaging (MRI), magnetic resonance angiography (MRA), mammography, x-ray, positron emission tomography (PET), PET-CT, ultrasound, or a combination thereof.

166. The method according to claim 162, wherein the interventional radiology procedure comprises stent placement via angiography or angioplasty, embolization to control bleeding, tumor embolization, tumor ablation, vertebroplasty, kyphoplasty, guided needle biopsy, guided breast biopsy, uterine artery embolization, feeding tube placement, venous access catheter placement, or a combination thereof.

167. The method according to claim 162, wherein the target compound comprises at least one of S-2-(3-aminopropylamino)ethyl phosphorothioic acid (amifostine, WR-2721), 2-[(aminopropyl)amino]ethanethiol (amifostine free thiol form, WR-1065), [2-[(aminopropyl) amino] ethanethiol] N,N,′-dithiodi-2,1-(ethanediyl) bis-1,3-propanediamine (amifostine disulfide form, WR-33278), S-[2-(3-methylaminopropyl)aminoethyl]phosphorothioate acid (phosphonol, WR-3689), 2-[3-(methylamino) propylamino] ethanethiol (WR-255591), S-3-(3-methylaminopropylamino) propyl phosphorothioic acid (WR-151327), 3-(3-methylaminopropylamino) propanethiol dihydrochloride (WR-151326), or a mixture thereof.

168. The method according to claim 162, wherein the composition includes the target composition at a dosage from about 10 mg/m² to about 600 mg/m².

169. The method according to claim 162, wherein the composition comprises an oral preparation or an injectable preparation.

170-240. (canceled)

241. The composition according to claim 154, wherein the composition includes the target composition at a dosage from about 25 mg/m² to about 225 mg/m².

242. The composition according to claim 154, wherein the composition includes the target composition at a dosage from about 600 mg/m² to about 2250 mg/m².

243. The method according to claim 162, wherein the composition is administered at a dosage from about 25 mg/m² to about 225 mg/m².

244. The method according to claim 162, wherein the composition is administered at a dosage from about 600 mg/m² to about 2250 mg/m².
